(19) United States
(12) Patent Application Publication
(10) Pub. No.: US 2009/0175933 A1
(43) Pub. Date: Jul. 9, 2009

(54) PREPARATION FOR THE TREATMENT OF DIARRHOEA

(75) Inventor: Michael David Levitt, Western Australia (AU)

Correspondence Address: ROTHWELL, FIGG, ERNST & MANBECK, P.C., 1425 K STREET, N.W., SUITE 800, WASHINGTON, DC, 20005, US

(73) Assignee: COLOCAPS PTY LTD, Subiaco, Western Australia (AU)

(21) Appl. No.: 12/089,448

(22) PCT Filed: Oct. 6, 2006
(86) PCT No.: PCT/AU2006/001469

§ 371 Date: Nov. 10, 2008

(30) Foreign Application Priority Data
Oct. 7, 2005 (AU) 2005905533

(51) Int. Cl. A61K 31/717; A61K 9/48; A61K 31/785; A61K 31/765; A61K 31/736; A61K 36/68; A61K 31/485; A61K 31/451; A61K 31/445; A61P 1/12

(52) U.S. Cl. 424/456; 424/78.35; 424/78.38; 424/195.18; 424/738; 514/22; 514/57; 514/282; 514/327; 514/330

(57) ABSTRACT

A preparation for the treatment of diarrhoea comprising a bulking agent and an anti-diarrhoeal agent wherein the anti-diarrhoeal agent is provided in the form of an anti-motility agent.

### CROSS REFERENCE TO RELATED APPLICATION(S)

This application is a 35 U.S.C. § 371 National Phase Entry Application from pct/au2006/001469, filed Oct. 6, 2006, and designating the United States. This application also claims the benefit of Australian Patent Application No. 2005905533 filed Oct. 7, 2005, the disclosure of which is incorporated herein in its entirety by reference.

### BACKGROUND OF THE INVENTION

1. Field of the Invention

The present invention relates to a preparation particularly suited to oral administration for the treatment of diarrhoea and other conditions associated with, or aggravated by, soft or liquid stool consistency.

2. Background Art

Bulking laxatives such as fibre supplements are widely recommended as laxatives largely because they are free of any tendency to induce tolerance (increasing dose requirements). They cause a mild acceleration of colonic transit (i.e. they have only a mild laxative action) and tend to encourage bowel actions to be bulky, formed and soft. They have the additional benefit of serving to lower serum cholesterol levels. They are considered to be “natural” and are held in high regard by the general population as being a safe and reliable group of products.

However, dietary fibre supplements are quite unpalatable and need to be combined with flavourings and then dissolved in water to be ingested. Too little fluid and the resulting mixture of fibre supplement and fluid is thick and difficult to ingest; too much fluid and the volume of the drink only highlights the indifferent taste. Further still, the texture of the drink can be somewhat granular regardless of the volume of water used, adding to the consumption problems. Even with the addition of flavourings, dietary fibre supplements remain difficult to consume as the flavourings do not completely mask the taste of the fibre.

Despite their tendency to speed up colonic transit, fibre supplements are also reputed to be of use in individuals suffering from diarrhoea or high stool frequency and soft stool consistency. These ailments are characteristically seen in the Irritable Bowel Syndrome or in individuals who have undergone extensive large bowel resection. Soft stool consistency is also common in the general population, especially amongst men. The proposed method of action of fibre supplements in these circumstances is by absorbing the liquid faeces and providing it with some bulk. Regrettably, this is a genuinely mild action and is often overwhelmed by the vigorous intestinal motility that underlies the high stool frequency and liquid stool consistency in these individuals. Perhaps unsurprisingly, the undeniable tendency of fibre supplements to speed up colonic transit can sometimes mean that these agents are disadvantageous in these clinical situations.

An alternative and yet more active agent for the treatment of individuals suffering from diarrhoea or high stool frequency and soft stool consistency are the anti-diarrhoeal agents. These drugs are designed to thicken stools and so to reduce diarrhoea. They will also firm up slightly soft stools. An example of such an agent is loperamide hydrochloride, generally referred to as loperamide, available commercially in 2 mg capsules and tablets under the trade mark Imodium®. Due to its mode of action, loperamide belongs to a class of anti-diarrhoeals known as anti-motility agents. Loperamide is a synthetic opiate-analogue with negligible systemic absorption, no ability to cross the blood-brain barrier and no known systemic side-effects. It has no tendency for the development of tolerance or addiction and is a potent anti-diarrhoeal by virtue of its tendency to slow down intestinal peristalsis by acting on both the cholinergic and prostaglandin phases of peristaltic activity. It is further able to enhance water re-absorption from the gastro-intestinal tract via mechanisms including opiate receptor stimulation, calcium channel blockade, calmodulin inhibition and a reduction in paracellular permeability. It also exerts an anti-secretory action by inhibition of secretory actions of numerous known stimuli for secretion such as Cholera Toxin and prostaglandins further reducing intra-intestinal fluid.

Difficulties encountered with the use of many anti-diarrhoeal agents relate to their very potency and it is a significant challenge when using anti-diarrhoeal agents to provide the correct dosage, as too much may induce severe constipation. Even low doses can be too strong and many individuals are unable to ingest, for example, even 2 mg of loperamide without becoming (or feeling as if they have become) constipated.

The preceding discussion of the background to the invention is intended to facilitate an understanding of the present invention. However, it should be appreciated that the discussion is not an acknowledgement or admission that any of the material referred to was part of the common general knowledge in Australia as at the priority date of the application.

Throughout the specification, unless the context requires otherwise, the word “comprise” or variations such as “comprises” or “comprising”, will be understood to imply the inclusion of a stated integer or group of integers but not the exclusion of any other integer or group of integers.

Throughout this specification, unless the context requires otherwise, the term diarrhoea refers to any intestinal disorder characterised by an increase in volume, fluidity or frequency of faecal discharge.

### DISCLOSURE OF THE INVENTION

In accordance with the present invention, there is provided a preparation for the treatment of diarrhoea comprising a bulking agent and an anti-diarrhoeal agent wherein the anti-diarrhoeal agent is provided in the form of an anti-motility agent.

The tendency of bulking agents to speed up colonic transit suggests that these agents would be disadvantageous in the treatment of diarrhoea. Advantageously though, the antagonistic modes of action of bulking agents and anti-diarrhoeal agents provides a preparation that reduces stool frequency but with the maintenance of a more satisfactory stool consistency. The modes of action of anti-diarrhoeal agents adapted to dehydrate stools and slow peristalsis and to enhance water re-absorption from the gastrointestinal tract and bulking agents adapted to retain water in stools and speed intestinal transit teaches away from their use together, yet the combination of the two agents deals effectively with the frequency and urgency of faecal discharge while reducing the tendency of anti-diarrhoeal agents alone to constipate the individual and produce a desiccated and unsatisfactory stool. That is, the propulsion of the stool is slowed by the anti-diarrhoeal agent while the bulking agent retains water and bulk to the stool. Further, the use of a bulking agent in combination with an anti-diarrhoeal agent reduces the likelihood of constipation that can result from the use of an anti-diarrhoeal agent alone.

Preferably, the preparation is provided in the form of a discrete unit comprising bulking agent and anti-diarrhoeal agent.

In one form of the invention, the discrete unit of bulking agent and anti-diarrhoeal agent is encapsulated in a pharmaceutically acceptable coating. Where the discrete unit of bulking agent and anti-diarrhoeal agent is encapsulated in a pharmaceutically acceptable coating, said unit may be provided in the form of a capsule or a cachet.

In a second form of the invention, the discrete unit is provided in the form of a tablet.

Preferably, each discrete unit contains a predetermined amount of bulking agent and anti-diarrhoeal agent.

Advantageously, the coating negates any unpalatable taste that may be associated with the bulking laxative and the anti-diarrhoeal agent, thereby improving palatability.

The bulking agent may be provided in the form of soluble or insoluble fibre. Soluble fibres are known to include pectins, gums, such as guar gum, mucilage, betaglucans, some forms of cellulose, brans, as well as ispaghula husk, psyllium, polycarbophil. Insoluble fibres are known to include polysaccharides, cellulose, lignin, brans, and pectin. In a preferred from of the invention, the bulking agent is provided in the form of guar gum.

In one form of the invention, the bulking agent may be dehydrated prior to encapsulation in a pharmaceutically acceptable coating or incorporation into a tablet.

Where the bulking agent is dehydrated, the coating preferably substantially inhibits absorption of atmospheric water, thereby substantially inhibiting rehydration of the bulking agent.

In the context of the present invention, the term dehydrated is intended to encompass partial removal of water from the bulking agent.

Preferably, the anti-diarrhoeal agent is provided in the form of an antimotility agent and may be selected from loperamide, loperamide hydrochloride (such as that sold under the trade marks Imodium® and Gastro-stop Loperamide®), loperamide oxide, diphenoxylate (such as that sold under the trade marks Lomotil®, Lofenoxal®, Lonox and Motofen), cholestyramine (such as that sold under the trade mark Questran®) and codeine. In the context of the present invention, the term loperamide shall be used to refer to loperamide, loperamide oxide and loperamide hydrochloride.

The coating of the preparation preferably substantially inhibits ingress of water.

Where the preparation is provided in the form of a pressed tablet, the preparation preferably comprises fillers and/or binders as known in the art.

Where the discrete units are provided in the form of tablets, each tablet of may be coated to substantially inhibit absorption of atmospheric water. Where the discrete units are provided in the form of cachets, the coating is provided in the form of the cachet, the bulking agent and the anti-diarrhoeal agent being contained therein. Where the discrete units are provided in the form of capsules, the coating is provided in the form of the capsule, the bulking agent and the anti-diarrhoeal agent being contained therein.

Where the discrete units are provided in the form of capsules, the capsules may be provided in the form of standard pharmaceutical capsules, such as those formed from gelatine or cellulose. Preferably, the capsules are provided in the form of soft gelatine capsules.

The discrete units each preferably comprise between about 250 mg to 1000 mg of bulking agent and between about 0.1 mg to about 5.0 mg of anti-diarrhoeal agent.

The final amount of bulking agent will be influenced by the size of the discrete unit, especially when provided in the form of a tablet or a capsule. The amount of anti-diarrhoeal will depend on the nature of the anti-diarrhoeal and will take into account the likelihood of over consumption and the side effects related thereto. It will be appreciated that given the potency of anti-diarrhoeal agents such as loperamide, great care should be taken to avoid over dose.

More preferably, the discrete units each comprise between about 300 mg to about 500 mg of bulking agent and between about 0.3 mg to about 0.5 mg of anti-diarrhoeal agent.

Most preferably, the discrete units each comprise about 500 mg of bulking agent and about 0.5 mg of anti-diarrhoeal agent.

In accordance with the present invention, there is further provided a method of treating a patient requiring such treatment, to cure diarrhoea, which comprises treating said patient with an effective amount of a preparation comprising a bulking agent and an anti-diarrhoeal agent to the patient.

The preparation of the present invention is particularly suitable for sufferers of irritable bowel syndrome or individuals who have undergone extensive large bowel resection.

The skilled addressee will appreciate that the dosage rate will depend on the individual's requirements. Without being limited by theory, it is expected that a daily dose of the preparation will comprise between about 3 to 6 g of bulking agent and 0.5 to 12 mg of anti-diarrhoeal agent.

Use of a bulking agent and an anti-diarrhoeal agent wherein the anti-diarrhoeal agent is provided in the form of an anti-motility agent for the preparation of a medicament for the treatment of diarrhoea

The present invention further provides a method for the preparation of a medicament for the treatment of a patient suffering from diarrhoea, using a preparation comprising a bulking agent and an anti-diarrhoeal agent.

In a preferred form of the invention, the patient is human.

### BEST MODE(S) FOR CARRYING OUT THE INVENTION

The best mode for performing the invention will now be described. It should be noted that the following description does not limit the scope of the invention as described in the preceding disclosure.

The recommended daily dose of loperamide is 0.5 to 12 mg, depending on the individual's requirements. The recommended daily dose of Benefiber® (78% guar gum) is 4 to 8 g, which translates to a dose of guar gum of approximately 3 to 6 g, depending on the individual's requirements. Using midpoints of the ranges, recommended daily dosages for loperamide are about 4 mg and guar gum about 4 g. On this basis, a preparation in accordance with the present invention could comprises loperamide and guar gum an a 1:1000 ratio.

Where the preparation is provided in the form of a capsule, it is expected that each capsule could comprises between about 300 to 500 mg of guar gum and 0.3 to 0.5 mg of loperamide and it would be necessary to consume between up to 20 of the smaller capsules per day and up to 10 of the larger capsules per day.

Modifications and variations such as would be apparent to a skilled addressee are deemed to be within the scope of the present invention.

1. A preparation for the treatment of diarrhoea comprising a bulking agent and an anti-diarrhoeal agent wherein the anti-diarrhoeal agent is provided in the form of an anti-motility agent.

2. A preparation for the treatment of diarrhoea according to claim 1, wherein the preparation is provided in the form of a discrete unit comprising bulking agent and anti-diarrhoeal agent.

3. A preparation for the treatment of diarrhoea according to claim 2, wherein the discrete unit is provided in the form of a capsule or a cachet.

4. A preparation for the treatment of diarrhoea according to claim 2, wherein the discrete unit is provided in the form of a tablet.

5. A preparation for the treatment of diarrhoea according to claim 2, wherein the discrete unit contains a predetermined amount of bulking agent and anti-diarrhoeal agent.

6. A preparation for the treatment of diarrhoea according to claim 1, wherein the bulking agent is provided in the form of soluble or insoluble fibre.

7. A preparation for the treatment of diarrhoea according to claim 6, wherein the bulking agent is selected from the group comprising pectins, gums, such as guar gum, mucilage, betaglucans, some forms of cellulose, brans, as well as ispaghula husk, psyllium, polycarbophil.

8. A preparation for the treatment of diarrhoea according to claim 6, wherein the bulking agent is guar gum.

9. A preparation for the treatment of diarrhoea according to claim 6, wherein the bulking agent is selected from the group comprising polysaccharides, cellulose, lignin, brans, and pectin.

10. A preparation for the treatment of diarrhoea according to claim 1, wherein the bulking agent is dehydrated.

11. A preparation for the treatment of diarrhoea according to claim 1, wherein the discrete unit is encapsulated in a pharmaceutically acceptable coating and the coating substantially inhibits absorption of atmospheric water.

12. A preparation for the treatment of diarrhoea according to claim 1, wherein the anti-motility agent is selected from the group comprising loperamide, diphenoxylate, cholestyramine and codeine.

13. A preparation for the treatment of diarrhoea according to claim 4, wherein the preparation comprises fillers and/or binders.

14. A preparation for the treatment of diarrhoea according to claim 4, wherein the tablet is coated to substantially inhibit absorption of atmospheric water.

15. A preparation for the treatment of diarrhoea according to claim 5, wherein the discrete unit is provided in the form of a cachet and the coating is provided in the form of the cachet, the bulking agent and the anti-diarrhoeal agent being contained therein.

16. A preparation for the treatment of diarrhoea according to claim 5, wherein the discrete unit is provided in the form of a capsule and the coating is provided in the form of the capsule, the bulking agent and the anti-diarrhoeal agent being contained therein.

17. A preparation for the treatment of diarrhoea according to claim 16, wherein the capsule is a standard pharmaceutical capsule, such as those formed from gelatine or cellulose.

18. A preparation for the treatment of diarrhoea according to claim 17, wherein the capsule is a soft gelatine capsules.

19. A preparation for the treatment of diarrhoea according to claim 2, wherein the discrete units each comprise between about 100 mg to about 1000 mg of bulking agent and between about 0.1 mg to about 2.0 mg of anti-diarrhoeal agent.

20. A preparation for the treatment of diarrhoea according to claim 2, wherein the discrete units each comprise between about 300 mg to about 500 mg of bulking agent and between about 0.3 mg to about 0.5 mg of anti-diarrhoeal agent.

21. A preparation for the treatment of diarrhoea according to claim 2, wherein the discrete units each comprise about 500 mg of bulking agent and about 0.5 mg of anti-diarrhoeal agent.

22. A method of treating a patient requiring such treatment, to cure diarrhoea, which comprises treating said patient with an effective amount of a preparation comprising a bulking agent and an anti-diarrhoeal agent wherein the anti-diarrhoeal agent is provided in the form of an anti-motility agent to the patient.

23. (canceled)

24. A method for the preparation of a medicament for the treatment of a patient suffering from diarrhoea, using a preparation comprising a bulking agent and an anti-diarrhoeal agent wherein the anti-diarrhoeal agent is provided in the form of an anti-motility agent.

25. (canceled)

26. (canceled)

27. A method of treating diarrhoea in a patient, which comprises treating said patient with an effective amount of a preparation comprising a bulking agent and an anti-diarrhoeal agent wherein the anti-diarrhoeal agent is provided in the form of an anti-motility agent to the patient.
